(19) United States
(12) Patent Application Publication
(10) Pub. No.: US 2018/0263954 A1
(43) Pub. Date: Sep. 20, 2018

(54) Sustained Release Cannabinoid Formulations

(71) Applicant: CannTab Therapeutics, Limited, Toronto (CA)

(72) Inventors: Jeff Scott Renwick, Thornhill (CA); Robert Lefler, Brantford (CA)

(21) Appl. No.: 15/923,095

(22) Filed: Mar. 16, 2018

Related U.S. Application Data
(63) Continuation-in-part of application No. 15/923,066, filed on Mar. 16, 2018.
Application No. 15/923,066 is a continuation-in-part of application No. 15/717,026, filed on Sep. 27, 2017.
(60) Provisional application No. 62/400,216, filed on Sep. 27, 2016.
(60) Provisional application No. 62/449,377, filed on Jan. 23, 2017.
(60) Provisional application No. 62/551,924, filed on Aug. 30, 2017.

(51) Int. Cl. A61K 31/352; A61K 31/05; A61K 9/14; A61K 9/48; A61K 36/185; A61K 9/16; A61K 9/50

(52) U.S. Cl. CPC A61K 31/352; A61K 31/05; A61K 9/148; A61K 9/4875; A61K 9/146; A61K 9/5089; A61K 9/145; A61K 9/4858; A61K 36/185; A61K 9/1694; A61K 9/4866

(57) ABSTRACT

The present invention provides modified release pharmaceutical composition comprising one or more natural or synthetic cannabinoids and one or more pharmaceutically acceptable excipients. More specifically, the invention relates to modified release pharmaceutical compositions comprising cannabinoids and a process for preparation thereof. The present invention also provides large scale batches of modified release pharmaceutical composition comprising one or more natural or synthetic cannabinoids and one or more pharmaceutically acceptable excipients.

### RELATED APPLICATIONS

This is a continuation-in-part of U.S. patent application Ser. No. 15/923,066 filed on Mar. 16, 2018 and is a continuation-in-part of U.S. patent application Ser. No. 15/717,026 filed on Sep. 27, 2017 which claims priority from U.S. provisional patent applications: Ser. No. 62/400,216, filed on Sep. 27, 2016; Ser. No. 62/449,377, filed on Jan. 23, 2017; and Ser. 62/551,924, filed on Aug. 30, 2017. The disclosures of all of the above applications are incorporated herein by reference in their entirety.

### FIELD OF THE INVENTION

The present invention relates to modified release pharmaceutical compositions comprising one or more natural or synthetic cannabinoids, one or more release modifying agent(s) and one or more pharmaceutically acceptable excipient(s). More specifically, the invention relates to modified release pharmaceutical compositions comprising cannabinoids and a process for preparation thereof. The invention also relates to production of large scale batches of modified release pharmaceutical compositions comprising cannabinoids and a process for preparation thereof.

### BACKGROUND OF THE INVENTION

Cannabinoids are a class of diverse chemical compounds that act on cannabinoid receptors on cells that repress neurotransmitter release in the brain. The most notable cannabinoid is the phytocannabinoid tetrahydrocannabinol (THC), the primary psychoactive compound of cannabis. Cannabidiol (CBD) is another major constituent of the plant. There are at least 85 different cannabinoids isolated from cannabis, exhibiting varied effects. From Wikipedia http://en.wikipedia.org/wiki/Tetrahydrocannabinol accessed May 5, 2015. All or any of these cannabinoids can be used in the present invention.

Synthetic cannabinoids encompass a variety of distinct chemical classes: the cannabinoids structurally related to THC, the cannabinoids not related to THC, such as (cannabimimetics) including the aminoalkylindoles, 1,5-diarylpyrazoles, quinolines, and arylsulfonamides, and eicosanoids related to the endocannabinoids. All or any of these cannabinoids can be used in the present invention.

Delta-9-Tetrahydrocannabinol (dronabinol) is a naturally occurring compound and is the primary active ingredient in marijuana. Marijuana is dried hemp plant Cannabis Sativa. The leaves and stems of the plant contain cannabinoid compounds (including dronabinol). Dronabinol has been approved by the Food and Drug Administration for the control of nausea and vomiting associated with chemotherapy and for appetite stimulation of patients suffering from wasting syndrome. Synthetic dronabinol is a recognized pharmaceutically active ingredient, but natural botanical sources of cannabis rather than synthetic THC are also known in the art. All or any of these cannabinoids can be used in the present invention.

Dronabinol is a light yellow resinous oil that is sticky at room temperature and hardens upon refrigeration. Dronabinol is insoluble in water and is formulated in sesame oil. It has a pKa of 10.6 and an octanol-water partition coefficient: 6,000:1 at pH 7. After oral administration, dronabinol has an onset of action of approximately 0.5 to 1 hours and peak effect at 2 to 4 hours. Duration of action for psychoactive effects is 4 to 6 hours, but the appetite stimulant effect of dronabinol may continue for 24 hours or longer after administration.

Dronabinol is the international nonproprietary name for a pure isomer of THC, (−)-trans-Δ⁹-tetrahydrocannabinol, which is the main isomer, and the principal psychoactive constituent, found in cannabis. Synthesized dronabinol is marketed as Marinol (a registered trademark of Solvay Pharmaceuticals).

Marinol is manufactured as a gelatin capsule containing synthetic delta-9-tetrahydrocannabinol (THC) in sesame oil. It is taken orally and is available in 2.5 mg, 5 mg and/or 10 mg dosages. Marinol is prescribed for the treatment of cachexia in patients with AIDS and for the treatment of nausea and vomiting associated with cancer chemotherapy in patients who have failed to respond adequately to conventional antiemetic treatments. Like other oils provided in gelatin dosage forms there is an urgent need for solid (powder and tablet) dosage forms of this drug as provided in the instant invention.

Despite FDA approval, it is almost universally accepted that medical marijuana has many benefits over Marinol and that by prohibiting the possession and use of natural cannabis and its cannabinoids, patients are unnecessarily restricted to use a synthetic substitute that lacks much of the therapeutic efficacy of natural cannabis. Sativex, is considered an improvement over Marinol. Sativex is an oral cannabis spray consisting of natural cannabinoid extracts, has greater bioavailability and is faster acting than oral synthetic THC. Of course oral sprays have numerous problems as a dosage form and Sativex has not been widely adopted as a replacement for medical marijuana. Why Marinol Is Not As Good As Real Marijuana Posted by Johnny Green on Mar. 5, 2012—see http://www.theweedblog.com/why-marinol-is-not-as-good-as-real-marijuana/accessed Sep. 18, 2016. Incorporated by reference in its entirety.

Marinol lacks several of the therapeutic compounds available in natural cannabis. Chemical compounds in cannabis, known as cannabinoids, are responsible for its numerous therapeutic benefits. Scientists have identified 66 naturally occurring cannabinoids. The active ingredient in Marinol, synthetic delta-9-tetrahydrocannabinol (THC), is an analogue of one such compound, THC. However, several other cannabinoids available in cannabis—in addition to naturally occurring terpenoids (oils) and flavonoids (phenols)—have also been clinically demonstrated to possess therapeutic utility. Many patients favor natural cannabis to Marinol because it includes these other therapeutically active cannabinoids. Why Marinol Is Not As Good As Real Marijuana Posted by Johnny Green on Mar. 5, 2012—see http://www.theweedblog.com/why-marinol-is-not-as-good-as-real-marijuana/accessed Sep. 18, 2016.

Cannabidiol (CBD) is a non-psychoactive cannabinoid that has been clinically demonstrated to have analgesic, antispasmodic, anxiolytic, antipsychotic, antinausea, and anti-rheumatoid arthritic properties. Clinical studies have shown CBD to possess anti-convulsant properties, particularly in the treatment of epilepsy. Natural extracts of CBD, when administered in combination with THC, significantly reduce pain, spasticity and other symptoms in multiple sclerosis (MS) patients unresponsive to standard treatment medications. CBD has been shown to be neuroprotective against glutamate neurotoxicity (i.e. stroke), cerebral infarction (localized cell death in the brain), and ethanol-induced neurotoxicity, with CBD being more protective against glutamate neurotoxicity than either ascorbate (vitamin C) or alpha-tocopherol (vitamin E). Clinical trials have also shown CBD to possess anti-tumoral properties, inhibiting the growth of glioma (brain tumor) cells in a dose dependent manner and selectively inducing apoptosis (programmed cell death) in malignant cells Why Marinol Is Not As Good As Real Marijuana Posted by Johnny Green on Mar. 5, 2012—see http://www.theweedblog.com/why-marinol-is-not-as-good-as-real-marijuana/accessed Sep. 18, 2016. Dosage formulations of CBD and other natural cannabinoids can also be formulated into solid dosage forms according to the present invention.

Additional cannabinoids possessing clinically demonstrated therapeutic properties include: cannabinol (anticonvulsant and anti-inflammatory activity); cannabichromine (anti-inflammatory and antidepressant activity); and cannabigerol (anti-tumoral and analgesic activity). Natural cannabis' essential oil components (terpenoids) exhibit anti-inflammatory properties and its flavonoids possess antioxidant activity. Emerging clinical evidence indicates that cannabinoids may slow disease progression in certain autoimmune and neurologic diseases, including multiple sclerosis (MS), Amyotrophic Lateral Sclerosis (Lou Gehrig's disease) and Huntington's Disease. Why Marinol Is Not As Good As Real Marijuana Posted by Johnny Green on Mar. 5, 2012—see http://www.theweedblog.com/why-marinol-is-not-as-good-as-real-marijuana/accessed Sep. 18, 2016. Dosage formulations of these cannabinoids can be formulated into solid dosage forms according to the present invention.

Oral ingestion of Marinol avoids the potential risks of smoking, however because of synthetic THC's poor bioavailability, only 5-20 percent of an oral dose ever reaches the bloodstream and the drug may not achieve peak effect until four hours after dosing. National Academy of Sciences, Institute of Medicine. 1999. Marijuana and Medicine: Assessing the Science Base. p. 203; L. Growing et al. 1998. Therapeutic use of cannabis: clarifying the debate. Drug and Alcohol Review. Moreover, because Marinol is metabolized slowly, its therapeutic and psychoactive effects may be unpredictable and vary considerably, both from one person to another, and in the same person from one episode of use to another. S. Calhoun et al. 1998. Abuse potential of dronabinol. Journal of Psychoactive Drugs. 30: 187-196; J. Morgan and L. Zimmer, Marijuana Myths, Marijuana Facts: A Review of the Scientific Evidence, p. 19. Thus there is a need for improved bioavailability dosage forms of natural and synthetic cannabinoids.

As a result of Marinol's slow onset and poor bioavailability, scientists are now in the process of developing a new formulation of pulmonary dronabinol, delivered with a pressurized metered dose inhaler. Medical News Today. “New synthetic delta-9-THC Inhaler offers safe, rapid delivery, Phase I study.” Apr. 17, 2005. Unlike oral synthetic THC, it's possible that pulmonary Marinol “could offer an alternative for patients when a fast onset of action is desirable.” Sativex, an oral cannabis spray consisting of natural cannabinoid extracts, has greater bioavailability and is faster acting than oral synthetic THC. Clinical trials comparing its bioavailability and time of peak onset compared to vaporized cannabis have not been performed, though anecdotal reports indicate that vaporized cannabis and its cannabinoids likely possess greater bioavailability and are faster acting than the Sativex spray. Thus there is a need for improved bioavailability, simple, inexpensive solid dosage forms of natural and synthetic cannabinoids.

U.S. Pat. No. 6,403,126 (incorporated herein by reference in its entirety) discloses methods of extracting and purifying cannabinoids from Cannabis using organic solvent.

An analog of dronabinol, nabilone. is available commercially.

US 20120231083 discloses a sustained release medicament which results in delivery of a therapeutic level of one or more cannabinoids during a clinically relevant therapeutic window. The therapeutic window is a longer window than provided by an immediate release medicament such as Marinol containing an equivalent amount of the cannabinoid. Oral administration of the present compositions provides therapeutic dosing while maintaining safe, side effect sparing, levels of a cannabinoid. The present invention also provides methods of treating cannabinoid-sensitive disorders.

US 20060257463 discloses a method of transmucosally delivering a cannabinoid to a subject in need of such treatment comprising the steps of: administering to the subject a transmucosal preparation containing the cannabinoid wherein said transmucosal preparation is made by incorporating an effective amount of the cannabinoid via hot-melt extrusion technology, hot-melt molding, admixing or a solvent cast technique into a film matrix or a reservoir containing the cannabinoid, and attaching said transmucosal preparation to the mucosa of the subject.

Pharmaceutical compositions comprising the cannabinoid active pharmaceutical ingredient, crystalline trans-(±)-Δ9-tetrahydrocannabinol, and formulations thereof are disclosed in WO 2006133941. The invention also relates to methods for treating or preventing a condition such as pain comprising administering to a patient in need thereof an effective amount of crystalline trans-(±)-Δ9-tetrahydrocannabinol. In specific embodiments, the crystalline trans-(±)-Δ9-tetrahydrocannabinol administered according to the methods for treating or preventing a condition such as pain can have a purity of at least about 98% based on the total weight of cannabinoids.

US 20140100269 A1 discloses oral cannabinoid formulations, including an aqueous-based oral dronabinol solution, that are stable at room or refrigerated temperatures and may possess improved in vivo absorption profiles with faster onset and lower inter-subject variability.

U.S. Pat. No. 8,632,825 discloses the use of a combination of cannabinoids, particularly tetrahydrocannabinol (THC) and cannabidiol (CBD), in the manufacture of a medicament for use in the treatment of cancer.

U.S. Pat. No. 6,630,507 discloses that cannabinoids have antioxidant properties. This property makes cannabinoids useful in the treatment and prophylaxis of wide variety of oxidation associated diseases, such as ischemic, age-related, inflammatory and autoimmune diseases. The cannabinoids are found to have particular application as neuroprotectants, for example in limiting neurological damage following ischemic insults, such as stroke and trauma, or in the treatment of neurodegenerative diseases, such as Alzheimer's disease, Parkinson's disease and HIV dementia. Nonpsychoactive cannabinoids, such as cannabidiol, are particularly advantageous to use because they avoid toxicity that is encountered with psychoactive cannabinoids at high doses useful in the method of the present invention.

U.S. Pat. No. 8,808,734 discloses stable, fast-acting liposomal and micelle formulations of cannabinoids or cannabinoid analogues.

U.S. Pat. No. 6,747,058 discloses stable composition for inhalation therapy comprising delta-9-tetrahydrocannabinol and semi-aqueous solvents.

### Dosage and Administration of Dronabinol from FDA Document NDA 18-651/S-021; 500012 Rev September 2004

Appetite Stimulation: Initially, 2.5 mg Dronabinol Capsules should be administered orally twice daily (b.i.d.), before lunch and supper. For patients unable to tolerate this 5 mg/day dosage, the dosage can be reduced to 2.5 mg/day, administered as a single dose in the evening or at bedtime. If clinically indicated and in the absence of significant adverse effects, the dosage may be gradually increased to a maximum of 20 mg/day, administered in divided oral doses. Caution should be exercised in escalating the dosage because of the increased frequency of dose-related adverse experiences at higher dosages.
Antiemetic: Best administered at an initial dose of 5 mg/m2, given 1 to 3 hours prior to the administration of chemotherapy, then every 2 to 4 hours after chemotherapy is given, for a total of 4 to 6 doses/day. Should the 5 mg/m2 dose prove to be ineffective, and in the absence of significant side effects, the dose may be escalated by 2.5 mg/m2 increments to a maximum of 15 mg/m2 per dose. Caution should be exercised in dose escalation, however, as the incidence of disturbing psychiatric symptoms increases significantly at maximum dose.

Despite all of the work on cannabinoids and dronabinol, there is a need in the art for simple, inexpensive, improved dosage forms that have an improved profile with faster onset, extended release profiles and lower inter-subject variability than currently available cannabinoid products.

Sticking is one of the most common problems of tablet making. It occurs when granules attach and stick to the faces of the punches instead of locking together to create a uniform tablet. Products with granules that are sensitive to compression—“sticky granules”—can form excellent tablets. But they are also prone to sticking to the punch faces and the problem worsens over the course of the production run because granules that are sensitive to compression will readily compact as they flow through the hopper and into the feed frame. The particles of the present invention solve this problem for cannabinoid formulations of resins, extracts and isolates.

Picking is a specific type of sticking in which particles stick within the letters and logos that are embossed or debossed on the faces of the compression tooling. Regardless whether it's sticking or picking, the result is a defective tablet. The particles of the present invention solve this problem for cannabinoid formulations of resins, extracts and isolates and allow for efficient encapsulation and tableting.

In the 1970s and 1980s there were almost no marketed drugs with less than 10 μg/ml solubility (10-100 μg/ml was considered low) (Solid Dispersions: New Approaches and Technologies in Oral Drug Delivery, Controlled Release Society; Rutgers, N.J. 2 Jun. 2009 Craig A. McKelvey Merck & Co., Inc. hereinafter “McKelvey”). Now it is estimated that more than 60% of Active Pharmaceutical Ingredients (API) in development have poor bioavailability due to low aqueous solubility (WO 2013040187 citing Manufacturing chemist, Mar. 24-25, 2010). At least partially as a result of advances in combinatorial chemistry and molecular screening methods for identifying potential drug candidates, an increasing number of insoluble drugs are being identified. Poor solubility of lead compounds results in ineffective absorption, which is an important part of the high clinical failure rate due to poor pharmacokinetics. Drugs with very low aqueous solubility usually have sizeable within and between subject pharmacokinetic variability making study design and the conduct of Phase I studies very challenging, the assessment of dose-response and exposure response relationships difficult, and resulting in difficult dose determination. Water insoluble drugs usually have high propensity for drug interactions at the absorption level, such as food interactions, and interactions with gastrointestinal “GI” prokinetic agents, especially if these drugs also have narrow therapeutic windows. There is an on-going need in the art for better formulation technologies for poorly soluble drugs (Jain et al. Asian J Pharm Clin Res, Vol 5, Suppl 4, 2012, 15-19).

The Biopharmaceutical Classification System (BCS) is a framework for classifying a drug substance on the basis of its equilibrium aqueous solubility and intestinal permeability. (Jain et al. Asian J Pharm Clin Res, Vol 5, Suppl 4, 2012, 15-19 hereinafter “Jain”) When combined with the in vitro dissolution characteristics of a drug product, the BCS takes into account three major factors: solubility, intestinal permeability and dissolution rate. These factors govern the rate and extent of oral drug absorption for immediate release solid oral dosage forms. The BCS defines four classes of drug substances based on their solubility and permeability characteristics.

| | High Solubility | Low Solubility |
|---|---|---|
| High Permeability | BCS Class I | BCS Class II |
| Low Permiability | BCS Class III | BCS Class IV |

A drug substance is considered highly soluble when the highest dose strength is soluble in 250 ml water over a pH range of 1 to 7.5. A drug is considered highly permeable when the extent of absorption in humans is determined to be 90% of an administered dose, based on the mass balance or in comparison to an intravenous dose (drug and metabolite). A drug product is considered to dissolve rapidly when 85% of the labeled amount of substance dissolves within 30 minutes, using USP apparatus I or II in a volume of 900 ml buffer solution. (Gothoskar A. V. Biopharmaceutical classification of drugs. Pharm Rev. 2005; 3:1.)

For BCS Class II drugs that have low bioavailability resulting from poor solubility and the inability to dissolve rapidly the selection of formulation is often a major hurdle preventing the development of a successful oral drug product. Certain technologies have recently been developed to aid in the formulation of these drugs including: salt formation, size reduction, co-solvency, pH manipulation, surfactant and micelle use, inclusion complexes, lipid formulations, and solid dispersions. Jain et al. Asian J Pharm Clin Res, Vol 5, Suppl 4, 2012, 15-19).

According to the “Intra-Agency Agreement Between the Eunice Kennedy Shriver National Institute of Child Health and Human Development (NICHD) and the U.S. Food and Drug Administration (FDA) Oral Formulations Platform—Report 1” dronabinol is a class 2 or class 4 drug with low solubility and unknown permeability. Thus it may be formulated in the same manner as a class 2 drug.

Absorption and distribution: Dronabinol capsules are almost completely absorbed (90 to 95%) after single oral doses. Due to the combined effects of first pass hepatic metabolism only 10 to 20% of the administered dose reaches the systemic circulation. FDA document NDA 18-651/S-021.

### Controlled Release Dosage Forms

Controlled-release formulations have been one of the major focuses in pharmaceutical research and development.

The advantages of controlled release products are well known in the pharmaceutical field. Sustained release drug formulations may be useful to reduce the frequency of drug administration (especially in the case of drugs with short compound half lives), improve patient compliance, reduce drug toxicity (local or systemic associated with high peak exposure), reduce drug level fluctuation in blood, stabilize medical condition with more uniform drug levels, reduce drug accumulation with chronic therapy, improve bioavailability of some drugs because of spatial control, and reduce total drug usage when compared with immediate release drugs.

Oral controlled release delivery systems should ideally be adaptable so that release rates and profiles can be matched to physiological and temporal requirements.

Mechanical devices aside, interaction between a drug and a polymeric material often forms the basis of controlled oral drug delivery. A polymer at certain concentrations in a solution imposes pathways for drug diffusion. Polymers that dissolve in or otherwise hydrate in aqueous media can alter the drug diffusion process in a time-dependent manner. For example, a commonly used material, hydroxypropyl methylcellulose (HPMC), which is water soluble, behaves as a swellable absorptive polymer in the limited volumes of aqueous media in the gastrointestinal tract. Drug dispersed in this polymer, as in monolithic tablets, diffuses through the viscous hydrated polymer at a rate dependent on the movement kinetics of the polymer chains. The faster these relax, the faster the diffusion rate.

Development of dosage form depends on chemical nature of the drug and polymers, the matrix structure, swelling, diffusion, erosion, the release mechanism and the in vivo environment.

Hydrophilic polymers like HPMC may also control drug release by erosion mechanisms. After consumption of the dosage form, the GI tract fluid encounters the dosage unit, causing the polymer to hydrate and swell. Weakened mechanical properties in the swollen state may cause the hydrated polymer to break away from the prime particle (compact or pellet). Drug release may therefore be controlled by a combination of diffusion and erosion. Such release mechanisms can apply to systems where drug is dispersed in or coated with polymer.

Extended release dosage forms of class 2 drugs often require expensive, difficult, and proprietary osmotic delivery systems such as Alza's Oros™ and Duros™ technologies. (See U.S. Pat. Nos. 4,612,008; 4,327,725; 4,765,989; and 4,783,337). Other technologies have been developed to exploit diffusion, erosion, and other physicochemical mechanisms and provide drug and disease-specific release profiles. Examples also include the release from a Contramid™ tablet controlled by the degree of crosslinking of high amylase starch.

Different hydrogels have been described for use in controlled release medicines, most of which are semi-synthetic or of natural origin. A few contain both synthetic and non-synthetic material. However, many of the systems require special process and production equipment, and in addition some of these systems are susceptible to variable drug release.

In another modified release approach, a solid dispersion comprising API with two different polymers is employed. JP Patent Application No. 2004-67606 discloses a tablet comprising fine pellets obtained by spraying a solution containing itraconazole, which is a poorly soluble drug, a water-soluble polymer and an enteric polymer, on a mixed powder of an excipient and a disintegrator, granulating and drying. Karel Six et al. (J. Pharm. Sci. 93, 124-131, 2004) discloses a solid dispersion composition of Itraconazole, a class II drug, Eudragit E100 and copovidone. The use of a combination of fast- and slow-dissolving polymers in solid dispersions compositions has resulted in increased physical stability and improved dissolution properties of itraconazole. In another approach, Hirasawa et al. (/. Pharm. Soc. of Japan, 124 (1), 19-23, 2004; Chem. Pharm. Bull. 52(2) 244-247, 2004; JP Patent Application No. 2001335483 A) disclose a solid dispersion comprising Nilvadipine (NIL)/Crospovidone (cl-PVP)/Methylcellulose (MC). US Patent Publication No. 20070248681 discloses a granule of a solid dispersion of a poorly soluble drug, a water-soluble polymer, an excipient and a disintegrator, wherein the content of the water-soluble polymer is 1 to 10% by weight and the content of the disintegrator is 15 to 50% by weight. A method for producing a tablet of a solid dispersion is also disclosed.

Water insoluble polymers can be used in extended drug release formulations. These include methacrylate- or acrylate-based polymers with low permeability.

Hydrophilic functional groups such as trimethylaminoethyl methacrylate can improve permeability and swellability in water thus altering release behaviors.

Various drug candidates such as diltiazem hcl, carbamazepine, metoprolol, oxprenolol, nifedipine, glipizide have been formulated as osmotic delivery systems. Problems with such osmotic delivery systems include the need for special equipment for making an orifice in the system; residence time of the system in the body varies with the gastric motility and food intake; such systems may cause irritation or ulcer due to release of saturated solutions of drug. Vol. 1 No. 7 2012. Online Available at www.thepharmajournal.com. THE PHARMA INNOVATION Vol. 1 No. 7 2012 www.thepharmajournal.com Page|116 Osmotic-Controlled Release Oral Delivery System: An Advanced Oral Delivery Form. Nitika Ahuja, Vikash Kumar, Permender Rathee.

The instant invention solves the problems associated with formulation cannabinoid drugs and provides for cannabinoid sustained release dosage forms in a technically and economically efficient and surprising manner.

In general, it would be advantageous if a cannabinoid containing capsule could be made available which does not suffer from the problems of expense and the need for smoking or “edible” dosage forms and for which the release profile can be controlled and optimized. None of the documents described above enable controlled release cannabinoid capsules or powders. There is a need for new cheap and stable dosage formulations, especially capsules, comprising an effective dose of cannabinoids or derivatives thereof. There is also a need for a stable cannabinoid powder and tablets.

Another aspect the invention provides a pharmaceutical or nutraceutical composition in the form of a capsules for oral administration comprising cannabinoid wherein said capsule is preferably formed from pharmaceutically or even nutraceutically acceptable pellets which provide controlled release profiles.

By “nutraceutical” is meant a composition that provides medical or health benefits, including the prevention and treatment of disease. Dietary supplements and natural health products are examples of nutraceuticals. In many places natural cannabinoids are considered nutraceuticals. Within the context of this invention it is understood that the term “drug” is used generically to include prescription and non-prescription pharmaceutical products as well as nutraceuticals including dietary supplements, natural health products, medicinal foods, drinks, candy bars with active ingredients and all other similar delivery methods whether approved or unapproved.

Viewed from another aspect the invention provides a pharmaceutical or nutraceutical capsule as hereinbefore described for use in the treatment or prophylaxis of all of the disorders that medical marijuana and dronabinol is used for at the present time.

As used herein, the term “drug” includes not only pharmaceuticals but also natural medicines, alternative medicines, and dietary supplements and generally refers to all forms of cannabinoids.

### DETAILED DESCRIPTION OF THE INVENTION

Extending drug release (“sustained release” or “controlled release”) from a dosage form can prolong its action and attenuate peak plasma levels, thereby obviating concentration-related side effects or optimize efficacy by matching systemic presence with other time-related effects. Sustained release drug forms can be achieved by embedding the drug in a matrix that prevents immediate release and delivers excipient at a desired rate consistent with absorption or disposition requirements. A wide variety of materials can be used to design the most appropriate release profile and provide a viable and consistent mode of manufacture. The present invention approaches this problem systematically and solves it in a unique way using particles of a very precise size and composition.

The present invention involves the use of cannabinoid particles that have been formulated to provide controlled release of the cannabinoid extract or isolate. The particles can be encapsulated, tableted, or provided in a powder-sachet dosage form. These particles are typically in the form of pellets or granules. Preferably, the diameter of the pellets ranges from about 0.05 to about 0.5 mm. Preferably, the average diameter of the particles ranges from 0.07 to 0.3 mm. Diameters referred to throughout the specification are average diameters. Although it is preferred that all particles are within the recited ranges, it is acceptable for minor or trace amounts of undersize or oversize particles to be present.

Surprisingly, it was found that cannabinoid containing particles of specific formulations and sizes minimize the sticking phenomena encountered during tablet compression or encapsulation. In one of its aspects, the present invention relates to a unit dosage form comprising a plurality of pellets having a diameter in the range of from 0.05 to 0.5 mm wherein each particle comprises a cannabinoid resin, extract or isolate, and at least one pharmaceutically acceptable excipient.

Such particles according to the present invention are stable and can be filled on fast running encapsulation machines without presenting the sticking and picking phenomena

Tablets and gelatin capsules of cannabinoids cannot easily be formulated from powder mix or by conventional wet granulation procedure due to picking and sticking phenomena during tablet compression or encapsulation. Thus, there was a need for extracts and isolates containing products and dosage forms which would be stable against moisture and heat during production and storage. Surprisingly, it was found that granules according to the present invention substantially between 0.05 mm and 0.5 mm in size are substantially easier to formulate into tablets and to encapsulate.

Prior to the methods and formulations of the present invention, tablets and hard gelatin capsules of CBDs and THC cannot easily be formulated from powder mix or by conventional wet granulation procedures due to picking and sticking phenomena during tablet compression or encapsulation. Thus, there was a need for extract containing products and dosage forms which would be stable against moisture and heat during production and storage.

Surprisingly, it was found that particles according to the present invention do not feature the sticking and picking phenomena and exhibit encapsulation and tabletting properties.

As discussed above, BCS Class II drugs present immense challenges for oral delivery, let alone attempts at zero order pharmacokinetics. In particular embodiments, the dosage form may provide a zero order release from about 1 hour to about 24 hrs after administration. In certain embodiments, the dosage form releases more than about 90% of the active agent in less than about 24 hrs. In particular embodiments, the dosage form may provide a zero order rate of release for at least a portion of the delivery period. In other embodiments, the dosage form may provide an ascending rate of release for at least a portion of the delivery period. In yet other embodiments, the dosage form may provide a fast initial rate of release followed by a slower rate of release and an ascending rate of release of the remaining active agent.

The sustained release formulations of cannabinoids of the present invention represent a significant improvement over existing formulations and delivery methods of cannabinoids.

The present invention involves a novel granulation method for formulating cannabinoids into a pellet matrix and subsequently into capsules.

The benefits of the invention include maintaining cannabinoids in a soluble, hydrophilic state in contact with body fluids.

The present invention provides a deceptively simple formulation solution to the problem of formulating modified release versions of cannabinoids involving a few simple ingredients combined in an extremely inventive and unique way. The present invention provides capsules and powders of cannabinoid formulations using a novel combination of silica gel, hydrogenated lecithin, glyceryl behenate, peg-8 caprylic/capric glycerides, hydroxypropylmethylcellulose, microcrystalline cellulose, colloidal silicon dioxide, and hydroxypropylcellulose.

### Cannabinoid Extract Resin

The cannabinoid extracts of the present invention can be extracted and formulated to provide a number of sustained release combinations useful in the present invention. Of particular interest are 100 percent THC tablets, 100% CBD tablets, 10:1 THC/CBD, 1:10 THC/CBD, and 50:50 THC/CBD although other variations of sustained release Pellets and tablets may be desirable in specific situations.

### Cyclodextrins

Cyclodextrins (sometimes called cycloamyloses) are a family of compounds made up of sugar molecules bound together in a ring (cyclic oligosaccharides).

Cyclodextrins are produced from starch by means of enzymatic conversion. They are used in food [Szente, L., & Szejtli, J. (2004). Cyclodextrins as food ingredients. Trends in Food Science & Technology, 15 (3-4), 137-142], pharmaceutical, [Stella, V., & He, Q. (2008). Cyclodextrins. Toxicologic Pathology, 36 (1), 30-42] drug delivery, [Laza-Knoerr, A. L., Gref, R., & Couvreur, P. (2010). Cyclodextrins for drug delivery. Journal of Drug Targeting, 18 (9), 645-656.] and chemical industries, as well as agriculture and environmental engineering.

Cyclodextrins are composed of 5 or more α-D-glucopyranoside units linked 1->4, as in amylose (a fragment of starch). The 5-membered macrocycle is not natural. Recently, the largest well-characterized cyclodextrin contains 32 1,4-anhydroglucopyranoside units, while as a poorly characterized mixture, at least 150-membered cyclic oligosaccharides are also known. Typical cyclodextrins contain a number of glucose monomers ranging from six to eight units in a ring, creating a cone shape:
α (alpha)-cyclodextrin: 6-membered sugar ring molecule
β (beta)-cyclodextrin: 7-membered sugar ring molecule
γ (gamma)-cyclodextrin: 8-membered sugar ring molecule
α- and γ-cyclodextrin are being used in the food industry.

All of these cyclodextrins can be employed in the present invention.

Cyclodextrins are able to form host-guest complexes with hydrophobic molecules given the unique nature imparted by their structure. As a result, these molecules have found a number of applications in a wide range of fields.

Because cyclodextrins are hydrophobic inside and hydrophilic outside, they can form complexes with hydrophobic compounds. Thus they can enhance the solubility and bioavailability of such compounds. This is of high interest for pharmaceutical as well as dietary supplement applications in which hydrophobic compounds shall be delivered.

Cyclodextrins can solubilize hydrophobic drugs in pharmaceutical applications, and crosslink to form polymers used for drug delivery. [Laza-Knoerr, A. L., Gref, R., & Couvreur, P. (2010). Cyclodextrins for drug delivery. Journal of Drug Targeting, 18 (9), 645-656. One example is Sugammadex, a modified γ-cyclodextrin which reverses neuromuscular blockade by binding the drug rocuronium. Other than the above-mentioned pharmaceutical applications, cyclodextrins can be employed in environmental protection: these molecules can effectively immobilise inside their rings toxic compounds, like trichloroethane or heavy metals, or can form complexes with stable substances, like trichlorfon (an organophosphorus insecticide) or sewage sludge, enhancing their decomposition.

Typical cyclodextrins are constituted by 6-8 glucopyranoside units, can be topologically represented as toroids with the larger and the smaller openings of the toroid exposing to the solvent secondary and primary hydroxyl groups respectively. Because of this arrangement, the interior of the toroids is not hydrophobic, but considerably less hydrophilic than the aqueous environment and thus able to host other hydrophobic molecules. In contrast, the exterior is sufficiently hydrophilic to impart cyclodextrins (or their complexes) water solubility.

The formation of the inclusion compounds greatly modifies the physical and chemical properties of the guest molecule, mostly in terms of water solubility. This is the reason why cyclodextrins have attracted much interest in many fields, especially pharmaceutical applications: because inclusion compounds of cyclodextrins with hydrophobic molecules are able to penetrate body tissues, these can be used to release biologically active compounds under specific conditions. In most cases the mechanism of controlled degradation of such complexes is based on pH change of water solutions, leading to the loss of hydrogen or ionic bonds between the host and the guest molecules. Alternative means for the disruption of the complexes take advantage of heating or action of enzymes able to cleave α-1,4 linkages between glucose monomers.

α-Cyclodextrin has been authorized for use as a dietary fiber in the European Union since 2008. In 2013 the EU commission has verified a health claim for alpha-cyclodextrin. The EU assessment report confirms that consumption of alpha-cyclodextrin can reduce blood sugar peaks following a high-starch meal. Weight loss supplements are marketed from alpha-cyclodextrin which claim to bind to fat and be an alternative to other anti-obesity medications.

Due to its surface-active properties, α-cyclodextrin can also be used as emulsifying fiber, for example in mayonnaise as well as a whipping aid, for example in desserts and confectionary applications.

β-cyclodextrins are the main ingredient in P&G's product Febreze which claims that the β-cyclodextrins “trap” odor causing compounds, thereby reducing the odor.

The cavity of the 7-membered β-cyclodextrin and 8-membered γ-cyclodextrin offer enough space even for comparatively large molecules, and are able to form the most stable complexes (Uekama, K., et al. (1983). Improvement of dissolution and absorption characteristics of benzodiazepines bycyclodextrin complexation. Int. J. Pharm., 10:1-15; Seo, H. et al. (1983) Enhancement of oral bioavailability of spironolactone by β- and γ-cyclodextrin complexations. Chem. Pharm. Bull., 31:286-291; Otagiri, M. et al. (1983) Inclusion complex formations of the anti-inflammatory drug flurbiprofen with cyclodextrins in aqueous solution and in solid state, Acta Pharm. Suec. 20:11-20.].

Alkylation of β-cyclodextrin functions with different substituents results in derivatives having a drastically increased aqueous solubility, while also preserving the complexing properties of the starting compound and allowing for solubilization [Muller B, Brauns U. Solubilization of drugs by modified β-cyclodextrins. Intl J Pharm 1985; 26: 77-88.] In addition, studies have shown a stabilizing effect on aqueous solutions, in which decomposition was delayed.

As mentioned above, the formation of inclusion compounds or “inclusion complexes” modifies the physical and chemical properties of the guest molecule, mostly in terms of water solubility, and allows hydrophobic molecules to penetrate body tissues and release biologically active compounds. Studies conducted on the use of indomethacin as a guest molecule, which normally undergoes controlled degradation by hydrolytic cleavage with a rate constant depending on the pH of the solution [Krasowska, H. (1974) Kinetics of indomethacin hydrolysis. Acta. Pharm. Jugoslay. 24:13-200.], was found to undergo delayed decomposition when it was solubilized by hydroxyethyl-β-cyclodextrin.

The silica gel is used herein as an adsorbant and solid carrier and should be selected for properties making it ideal for use with lipid formulations; able to adsorb large amounts of oils with a resulting density and flowability that is useful for maximum loading into tablets. It is also desirable that the oil will release from the silica gel without the use of additional surfactants.

Lecithin is a naturally occurring mixture of the diglycerides of stearic, palmitic, and oleic acids, linked to the choline ester of phosphoric acid, commonly called phosphatidylcholine. Hydrogenated Lecithin is the product of controlled hydrogenation of Lecithin. Bilayers of these phospholipids in water may form liposomes, a spherical structure in which the acyl chains are inside and not exposed to the aqueous phase. Lecithin and Hydrogenated Lecithin are used in a large number of cosmetic formulations as skin conditioning agents-miscellaneous and as surfactant-emulsifying agents. Hydrogenated Lecithin is also used as a nonsurfactant suspending agent. Lecithin is virtually nontoxic in acute oral studies, short-term oral studies, and subchronic dermal studies in animals. Lecithin is not a reproductive toxicant, nor is it mutagenic in several assays. Fiume Z. Int J Toxicol. 2001; 20 Suppl 1:21-45.

Soy lecithin one of the most widely used food additives on the market today. It is used as an emulsifier. It helps to emulsify numerous foods, even unlikely emulsions such as chocolate. In chocolate, lecithin stabilizes the cocoa butter fat so it doesn't separate from the moisture, cocoa solids and dairy.

Lecithin also extends shelf life by stabilizing emulsions, and it also reduces “stickiness” and is often used as a “releasing agent.”

Chemically, glyceryl behenate is a mixture of various esters of behenic acid and glycerol (glycerides). One example is Campitrol 888. The mixture predominately contains the diester glyceryl dibehenate. 21 C.F.R. 184.1328. Glyceryl behenate is a tablet and capsule lubricant and a lipidic coating excipient. It has been used for the encapsulation of various drugs such as retinoids. It has also been used as a matrix-forming agent for the controlled release of water-soluble drugs and as a lubricant in oral solid dosage formulations. It is also used widely as ingredient for preparation of lipidic nano-particles such as solid lipid nanoparticles (SLN) and nanostructured lipid carriers (NLC). Handbook of pharmaceutical excipient, 5th edition.

Peg-8 caprylic/capric glycerides (Labrasol) is a polyethylene glycol derivative of a mixture of mono-, di-, and triglycerides of caprylic and capric acids with an average of 6 or 8 moles of ethylene oxide. It is used in the present invention as an emulsifying agent. A preferred form is caprylocaproyl macrogol-8 glycerides, a non-ionic water dispersible surfactant composed of polyethylene glycol (PEG) esters, a glyceride fraction, and free PEG. This form is able to self-emulsify on contact with aqueous media to form a fine micro-emulsion. It is a solubilizer and wetting agent: its surfactive power improves the solubility and wettability of active pharmaceutical ingredients in vitro and in vivo. See for example, http://www.gattefosse.com.

The preparation of cannabinoid isolates can be completed by many well-known methods. See U.S. Pat. No. 2,304,669A, Adams, Roger, D. C. Pease, and J. H. Clark. “Isolation of cannabinol, cannabidiol and quebrachitol from red oil of Minnesota wild hemp.” Journal of the American Chemical Society 62.8 (1940): 2194-2196, Ben-Shabat, Shimon, et al. “New cannabidiol derivatives: synthesis, binding to cannabinoid receptor, and evaluation of their antiinflammatory activity.” Journal of medicinal chemistry 49.3 (2006): 1113-1117., and Lago-Fernandez, Ana, et al. “New Methods for the Synthesis of Cannabidiol Derivatives.” Methods in enzymology. Vol. 593. Academic Press, 2017. 237-257. for examples and an overview of the preparation of cannabinoid isolates.

One advantage of cannabinoid isolates is the ability to isolate the desired cannabinoid for use in pharmaceutical compositions, such as those which form the instant invention.

An advantage of using cannabinoid isolates instead of cannabinoid resin is that cannabinoid isolates that are pure CBD, or nearly pure CBD, can be formed. Cannabinoid isolates comprising 100% CBD, or nearly 100% CBD, can be formed by a process which removes THC and other cannabinoids from a plant extract, cannabinoid resin, or other cannabinoid extract.

Therefore, cannabinoid isolates can be used in the instant invention to form compositions which are high in CBDs. Cannabinoid isolates which comprise 100% CBD, or nearly 100% CBD, can be utilized to prepare 100% CBD tablets, which are particularly of interest.

Another advantage of the use of Cannabinoid isolates in compositions of the present invention is that a carrier oil, such as sesame oil, is not required for formulation.

In each of the following examples cannabinoid isolates may be advantageously substituted for cannabinoid resin.

The preparation of cannabinoid isolates can be completed by many well-known methods. See U.S. Pat. No. 2,304,669A, Adams, Roger, D. C. Pease, and J. H. Clark. “Isolation of cannabinol, cannabidiol and quebrachitol from red oil of Minnesota wild hemp.” Journal of the American Chemical Society 62.8 (1940): 2194-2196, Ben-Shabat, Shimon, et al. “New cannabidiol derivatives: synthesis, binding to cannabinoid receptor, and evaluation of their anti-inflammatory activity.” Journal of medicinal chemistry 49.3 (2006): 1113-1117., and Lago-Fernandez, Ana, et al. “New Methods for the Synthesis of Cannabidiol Derivatives.” Methods in enzymology. Vol. 593. Academic Press, 2017. 237-257. for examples and an overview of the preparation of cannabinoid isolates.

One advantage of cannabinoid isolates is the ability to isolate the desired cannabinoid for use in pharmaceutical compositions, such as those which form the instant invention.

An advantage of using cannabinoid isolates instead of cannabinoid resin is that cannabinoid isolates that are pure CBD, or nearly pure CBD, can be formed. Cannabinoid isolates comprising 100% CBD, or nearly 100% CBD, can be formed by a process which removes THC and other cannabinoids from a plant extract, cannabinoid resin, or other cannabinoid extract.

Therefore, cannabinoid isolates can be used in the instant invention to form compositions which are high in CBDs. Cannabinoid isolates which comprise 100% CBD, or nearly 100% CBD, can be utilized to prepare 100% CBD tablets, which are particularly of interest.

Another advantage of the use of Cannabinoid isolates in compositions of the present invention is that a carrier oil, such as sesame oil, is not required for formulation.

In a preferable example of compositions of the instant invention, the compositions use a cannabinoid isolate which is water soluble.

### EXAMPLES

### Example 1

### Ingredients and Amounts Useful for Dosage Units of 25 mg of Cannabinoid Pellets

Pellets—229.0 mg of Pellets

| | | |
|---|---|---|
| beta-cyclodextrin | 150.0 | mg |
| Sesame Oil | 25.0 | mg |
| Cannabinoid Resin | 25.0 | mg |
| Compritol 888 | 4.0 | mg |
| Soy Lecithin | 2.5 | mg |
| Labrasol | 22.5 | mg |

### Example 2

### Pellet Formulation Methods

The pellet formulations according to the present example may be prepared as follows:
1. mix cyclodextrin with water for approximately 2.5 hours to form a slurry;
2. mix a cannabinoid resin and sesame oil together at a temp of about 60° C. until a uniform mixture is obtained;
3. add the uniform mixture or resin and oil to the cyclodextrin slurry and mix for about 1 hour;
4. mix soy lecithin and water together at a temperature of about 60° C., until a uniform slurry mixture is obtained;
5. slowly sprinkle the glyceryl behenate on to the resin, cyclodextrin mixture obtained in step 3 and mix for about 15 minutes;
6. slowly add the soy lecithin slurry to the mixture obtained in step 5 while increasing the mixer speed to achieve a uniform mixture;
7. slowly add Labrasol to the mixture obtained in step 6 while maintaining the uniform mixture;
8. continue mixing until a uniform mixture is obtained and being careful to not over mix;
9. transfer the mixture to stainless steel (or other suitable) trays;
10. place in an oven and dry at about 70° C. until the moisture content is less than 2.0% to form Pellets; and
11. screen the dry mixture using mesh screens of 30 followed by a mesh of 270 to obtain particles substantially between 0.05 mm and 0.5 mm in size.

### Example 3

### Branded Ingredients Useful for 25 mg of Cannabinoid Pellets

Pellets

| | | |
|---|---|---|
| beta-cyclodextrin | 150.0 | mg |
| Sesame Oil | 25.0 | mg |
| Cannabinoid Resin | 25.0 | mg |
| Compritol 888 | 4.0 | mg |
| Soy Lecithin | 2.5 | mg |
| Labrasol | 22.5 | mg |

### Example 4

### Ingredients Useful for Preparing Larger Scale Cannabinoid Pellet Batches

Pellets—

| | | |
|---|---|---|
| beta-cyclodextrin | 1.5 | kg |
| Sesame Oil | 0.250 | kg |
| Cannabinoid Resin | 0.250 | kg |
| Compritol 888 | 0.050 | kg |
| Soy Lecithin | 0.050 | kg |
| Labrasol | 0.230 | kg |

### Example 6

### Pellet Formulation Methods

The formulation according to the present example may be prepared as follows:
1. mix cyclodextrin with water for approximately 2.5 hours to form a slurry;
2. mix a cannabinoid resin and sesame oil together at a temp of about 60° C. until a uniform mixture is obtained;
3. add the uniform mixture or resin and oil to the cyclodextrin slurry and mix for about 1 hour;
4. mix soy lecithin and water together at a temperature of about 60° C., until a uniform slurry mixture is obtained;
5. slowly sprinkle the glyceryl behenate (Comp888) on to the resin, cyclodextrin mixture obtained in step 3 and mix for about 15 minutes;
6. slowly add the soy lecithin slurry to the mixture obtained in step 5 while increasing the mixer speed to achieve a uniform mixture;
7. slowly add Labrasol to the mixture obtained in step 6 while maintaining the uniform mixture;
8. continue mixing until a uniform mixture is obtained and being careful to not over mix;
9. transfer the mixture to stainless steel (or other suitable) trays;
10. place in an oven and dry at about 70° C. until the moisture content is less than 2.0% to form Pellets; and
11. screen the dry mixture using mesh screens of 30 followed by a mesh of 270 to obtain particles substantially between 0.05 mm and 0.5 mm in size.
Surprising the amounts of glyceryl behenate and soy lecithin are crucial to control as too little will result in very long drying times for the pellets and a loss of efficiency.

### Example 7

### Ingredients Useful for Preparing Cannabinoid Pellets using a Cannabinoid Isolate

Granules

| | | |
|---|---|---|
| Beta-cyclodextrin | 0.3 | kg |
| Purified water | 0.945 | kg |
| Cannabinoid Isolate | 0.070 | kg |
| Compritol 888 | 0.010 | kg |
| Soy Lecithin | 0.020 | kg |
| Labrasol | 0.046 | kg |

In each of the foregoing examples cannabinoid isolates may be advantages substituted for cannabinoid resin or extract.

A further aspect of the invention are pharmaceutical preparations or compositions for oral administration, comprising the particles. They can be simply filled in a PVC container from which the particles can be taken with a dosing spoon. Other oral dosage forms are sachets in which the particles are filled, alone or together with appropriate excipients, such as skim milk powder, microcrystalline cellulose, sodium carboxymethylcellulose and talc, to form a powder for reconstitution. Another possibility is to embed the particle in a matrix excipient, for example, microcrystalline cellulose, followed by compression to tablets, particularly chewable tablets. The particle, can also be filled in capsules, for example, hard gelatin capsules.

As will be immediately obvious, some of the steps may be carried out simultaneously or in a different order, such variations are included in the present invention.

All publications mentioned above are hereby specifically incorporated herein by reference in full for the teachings for which they are cited. The examples and claims of the present invention are not limiting. Having read the present disclosure, those skilled in the art will readily recognize that numerous modifications, substitutions and variations can be made to the description without substantially deviating from the invention described herein. Such modifications, substitutions and variations constitute part of the invention described herein.

1. A composition comprising cannabinoid plus sesame oil, a cyclodextrin, glyceryl behenate, lecithin, and labrasol wherein the composition is in the form of pellets which pellets are substantially between 0.05 mm and 0.5 mm in size.

2. The composition according to claim 1 wherein the cannabinoid is selected from the group consisting of an extract, an isolate and a resin.

3-4. (canceled)

5. The composition according to claim 1 further comprising a capsule.

6. A composition according to claim 1 wherein the cannabinoid comprises a tetrahydrocannabinol.

7. A composition according to claim 1 wherein the cannabinoid comprises a CBD.

8. A composition according to claim 1 wherein the cannabinoid comprises a natural extract of Cannabis Sativa.

9. A composition according to claim 5 comprising about 25 mg, 15 mg, 10 mg, 5 mg, or 2.5 mg of cannabinoid per capsule.

10. A composition according to claim 9 wherein the composition comprises about 25 mg of cannabinoid per capsule.

11-12. (canceled)

13. A composition according to claim 9 wherein the composition comprises about 5 mg of cannabinoid per capsule.

14. A composition according to claim 9 wherein the composition comprises about 2.5 mg of cannabinoid per capsule.

15. A composition according to claim 6 wherein the cannabinoid further comprises a CBD.

16. A composition according to claim 15 wherein the cannabinoid has a THC to CBD ratio of about 10:1 to 1:10.

17. A composition according to claim 16 wherein the THC to CBD ratio is about 50:50.

18. A method of formulating a dosage form comprising forming pellets by:
i) mixing a cannabinoid with a non-toxic organic solvent to form a slurry;
ii) mixing a cyclodextrin with water;
iii) combining the slurry from i) and the mixture from ii) to form a uniform slurry;
iv) mixing lecithin with water until a uniform mixture is obtained;
v) sprinkling glyceryl behenate into the mixture from step iii);
vi) slowly add the lecithin mixture from step iv) to the slurry formed in step v);
vii) adding slowly polyethylene glycol-8 caprylic/capric glycerides to the mixture of step vi);
viii) mixing until a uniform mixture is obtained and being careful to not over mix;
ix) transferring the mixture to stainless steel trays;
x) placing the trays to an oven and drying at about 70° C. until the moisture content of the mixture is less than 2.0% to form a dry mixture of pellets; and
xi) screening the dry mixture using mesh screens of 30 followed by a mesh of 270 to obtain particles substantially between 0.05 mm and 0.5 mm in size.

19. A method of formulating a drug comprising:
a. mixing cyclodextrin with water for approximately 2.5 hours to form a slurry;
b. mixing a cannabinoid resin and sesame oil together at a temp of about 60° C. until a uniform mixture is obtained;
c. adding the uniform mixture or resin and oil to the cyclodextrin slurry and mix for about 1 hour;
d. mixing soy lecithin and water together at a temperature of about 60° C., until a uniform slurry mixture is obtained;
e. slowly sprinkling the glyceryl behenate on to the resin, cyclodextrin mixture obtained in step 3 and mix for about 15 minutes;
f. slowly adding the soy lecithin slurry to the mixture obtained in step 5 while increasing the mixer speed to achieve a uniform mixture;
g. slowly adding Labrasol to the mixture obtained in step 6 while maintaining the uniform mixture;
h. mixing the uniform mixture obtained in step g for about an additional 30;
i. transferring the mixture to stainless steel trays;
j. placing the trays in an oven and drying at about 70° C. until the moisture content is less than 2.0% to form pellets; and
k. filling capsules with a desired amount of said pellets.

20-21. (canceled)

22. A composition according to claim 17 comprising about 25 mg, 15 mg, 10 mg, 5 mg, or 2.5 mg of cannabinoid per capsule.

23-29. (canceled)

30. The composition of claim 1, said composition comprising a plurality of pellets having a diameter in the range of from about 0.05 mm to about 0.5 mm, and at most only a trace amount of pellets outside this range, each pellet comprising about 10% by weight of cannabinoid; and at least one pharmaceutically acceptable excipient.

31. The composition of claim 30, wherein the particles have a diameter of from about 0.07 mm to about 0.3 mm.

32. The composition of claim 30 wherein the composition further comprises at least 40 to about 60% cyclodextrin.

33. The composition of claim 32 wherein the ratio of cyclodextrin to cannabinoid extract is about 6 to 1.
